(19) United States
(12) Patent Application Publication
(10) Pub. No.: US 2017/0087001 A1
(43) Pub. Date: Mar. 30, 2017

(54) COMPRESSION LEG SUPPORT

(71) Applicant: M.J. Fullerton, Pryor, OK (US)

(72) Inventor: M.J. Fullerton, Pryor, OK (US)

(21) Appl. No.: 15/186,990

(22) Filed: Jun. 20, 2016

Related U.S. Application Data
(63) Continuation-in-part of application No. 14/959,897, filed on Dec. 4, 2015.
Application No. 14/959,897 is a continuation-in-part of application No. 14/546,191, filed on Nov. 18, 2014, now Pat. No. 9,204,985.

(51) Int. Cl. A61F 5/01; A61F 5/34; A61F 5/058

(52) U.S. Cl. CPC A61F 5/0195; A61F 5/0102; A61F 5/012; A61F 5/0585; A61F 5/0111; A61F 5/0106; A61F 5/34

(57) ABSTRACT

A leg support that attaches to a user's leg and extends below the user's foot, where stepping on the portion below the user's foot causes the portion around the user's upper leg to tighten. This allows the leg support and the user's leg to bear the user's weight with the user's foot remaining untouched. When the user takes his or her weight off of the leg support, the portion around the user's upper leg loosens for the user's comfort. The leg support insures that there is zero pressure on the bottom of the user's foot and all of the user's weight and vertical shear pressure is converted into horizontal compression pressure around the user's leg.

### CROSS REFERENCE

This is a continuation-in-part of U.S. patent application Ser. No. 14/959,897 filed Dec. 4, 2015 and U.S. patent application Ser. No. 14/425,191 filed Nov. 18, 2014, now U.S. Pat. No. 9,204,985, which are incorporated herein by reference.

### BACKGROUND OF THE INVENTION

Field of the Invention

This invention relates generally to a leg support, and more particularly, but not by way of limitation, to a leg support that allows a user to walk without putting any pressure on the user's foot by transforming a portion of the vertical shear force to horizontal compressive force around the user's upper leg.

Description of the Related Art

There are many circumstances that require a person to keep all pressure off his or her foot or lower leg, including broken bones, sprains, tears, cuts, bruises, abrasions, burns, and injuries due to diabetes. Typically, this necessitates the use of crutches or a wheelchair, both of which are cumbersome and limit the mobility of the user. Many people in such circumstances opt for a device that allows them to continue walking, such as a boot or other device designed to minimize the impact on the injured area. These devices do not completely relieve the pressure on the injured area, however, potentially prolonging recovery time and increasing the user's pain.

For example, diabetes can cause a variety of injuries to a person's foot, including ulcers. The standard treatment for a foot ulcer is a boot with a pad surrounding the ulcer, with an opening cut into the pad at the location of the ulcer to prevent impact to the ulcer itself. Every step, however, impacts the skin adjacent the ulcer, pulling the healing tissue. This is not only painful, but also delays healing. Unhealed ulcers can lead to amputation. The best course of treatment is to keep any and all pressure from the entire foot, but many people do not want or cannot tolerate the resultant disruption to their lives caused by such lack of mobility.

A further consideration is that a person's knee needs to bend to allow the person to sit or stand without assistance. May braces, however, are rigid at the knee and do not provide for bending. Getting up from a seated position is very difficult, if not impossible, with a rigid, straight leg brace.

Based on the foregoing, it is desirable to provide a leg support that transfers all of the pressure of walking to the leg rather than the foot.

It is further desirable for such a leg support to allow a user to walk without putting any pressure on the user's foot.

It is further desirable for such a leg support not to touch the bottom of a user's foot at all.

It is further desirable for a user to be able to take such a leg support off and on without having to insert his or her foot through any openings.

It is further desirable for such a leg brace not to inhibit the bending of the user's knee to allow the user to get up from a seated position.

It is further desirable to allow the user to finely adjust the pressure of the leg support to maximize comfort and pressure for all positions: standing, seated, ad walking. It is desirable for the user to have total control of the pressure of the leg support on the user's leg. It is further desirable for such a leg brace to allow small adjustments and to hold those adjustments without constant readjustment as the leg support is used.

It is further desirable for such a leg support to have a knee hinge that bends in an arch rather than at a 90° angle, if the leg support extends above the knee.

It is further desirable for the knee hinge of the leg support to bend or flex in only one plane or direction, if the leg support extends above the knee.

It is further desirable for such a leg support to provide confidence in a user to facilitate increased usage.

It is further desirable for such a leg support to be simple and consistent in usage in design, allowing for mass production and affording removal of leg pain and pressure on leg, knee, ankle, foot, and sole of foot without major cost of production.

It is further desirable for such a leg support to allow a user to put the leg support on and take the leg support off easily and to quickly and accurately adjust all pressure and clearance of the sole of the foot.

### SUMMARY OF THE INVENTION

In general, in a first aspect, the invention relates to a leg support comprising an upper assembly, a bracket, and a force conveyance assembly. The upper assembly may comprise a front half, a back half, and a bladder located within the front half or the back half. The bracket may comprise a right leg with a top connected to the upper assembly and a bottom; a left leg with a top connected to the upper assembly and a bottom; and a cross piece connecting the bottom of the right leg to the bottom of the left leg, where the cross piece has a bottom. The force conveyance assembly may comprise a bladder attached to the bottom of the cross piece, where the bladder in the force conveyance assembly is in fluid communication with the bladder in the upper assembly such that pressing the bottom of the cross piece against a surface forces air within the bladder of the force conveyance assembly into the bladder in the upper assembly.

The front half and the back half of the upper assembly may each be elongate and have a semi-circular cross section such that the front half and the back half are capable of connecting to form a hollow cylinder or a hollow conical frustum. In other words, the upper assembly may have a hollow cylindrical or hollow conical frustum shape. The front half and the back half of the upper assembly may each have a right side and a left side. The right leg of the bracket may comprise a front right leg and a back right leg, where the front right leg is connected to the right side of the front half of the upper assembly and the back right leg is connected to the right side of the back half of the upper assembly. Similarly, the left leg of the bracket may comprise a front left leg and a back left leg, where the front left leg is connected to the left side of the front half of the upper assembly and the back left leg is connected to the left side of the back half of the upper assembly. The cross piece may comprise a front cross piece connecting the bottom of the front right leg to the bottom of the front left leg and a back cross piece connecting the bottom of the back right leg to the bottom of the back left leg, where the front cross piece is hingedly connected to the back cross piece.

The front half of the upper assembly and the back half of the upper assembly may each have an interior surface. The upper assembly may further comprise a liner substantially covering the interior surface of the front half of the upper assembly and a liner substantially covering the interior surface of the back half of the upper assembly, where the bladder is intermediate of either the interior surface of the front half and the liner substantially covering the interior surface of the front half or the interior surface of the back half and the liner substantially covering the interior surface of the back half. The liners may comprise hardened expanding foam.

The force conveyance assembly may further comprise tubing fluidly connecting the bladder of the force conveyance assembly to the bladder of the upper assembly. The force conveyance assembly may further comprise a bulb and a release valve fluidly connected to the bladder of the force conveyance assembly such that air pressure within the bladder is capable of being adjusted via the bulb and release valve.

### BRIEF DESCRIPTION OF THE DRAWINGS

FIG. 1 is a side view of the compression leg support in place on a user's upper leg, with the compression leg support in a non-compressed position;

FIG. 2 is a side view of the compression leg support in place on a user's upper leg, with the compression leg support in a compressed position;

FIG. 3 is a side view of the compression leg support in place on a user's upper leg, with the compression leg support in a bent position;

FIG. 4 is a back view of the compression leg support in a non-compressed position;

FIG. 5 is a front view of the compression leg support in a non-compressed position;

FIG. 6 is a close-up front view of the upper portion of the compression leg support in a non-compressed position;

FIG. 7A is a close-up side view of the outside of the hinge assembly;

FIG. 7B is a close-up side view of the outside of the hinge assembly in a bent position;

FIG. 8A is a close-up side view of the inside of the hinge assembly;

FIG. 8B is a close-up side view of the inside of the hinge assembly in a bent position;

FIG. 9 is a top view of the compression leg support;

FIG. 10 is a top view of the compression leg support in an open position;

FIG. 11 is an exploded top view of the compression leg support in an open position;

FIG. 12 is a side view of the compression leg support in place on a user's lower leg, with the compression leg support in a non-compressed position;

FIG. 13 is a side view of the compression leg support in place on a user's lower leg, with the compression support in a compressed position;

FIG. 14 is a back view of the compression leg support in a non-compressed position;

FIG. 15 is a front view of the compression leg support in a non-compressed position;

FIG. 16 is a side cut away view of the compression leg support in a non-compressed position;

FIG. 17 is an exploded view of the compression leg support;

FIG. 18 is a top view of the lower leg wrap;

FIG. 19 is a top exploded view of the lower leg wrap;

FIG. 20 is a side view of an air pressure version of the compression leg support in place on a user's lower leg, with the compression leg support in a non-compressed position;

FIG. 21 is a side view of the air pressure version of the compression leg support in place on a user's lower leg, with the compression leg support in a compressed position;

FIG. 22 is a back view of the air pressure version of the compression leg support in a non-compressed position;

FIG. 23 is a front view of the air pressure version of the compression leg support in a non-compressed position;

FIG. 24 is a schematic view of the air pressure version of the compression leg support in the first pumping stage;

FIG. 25 is a schematic view of the air pressure version of the compression leg support in the second compressing stage;

FIG. 26 is a schematic view of the air pressure version of the compression leg support in the third decompressing stage;

FIG. 27 is a top view of the air pressure version of the lower leg wrap;

FIG. 28 is an exploded top view of the air pressure version of the lower leg wrap;

FIG. 29 is a schematic view of a hydraulic version of the compression leg support in the first pumping stage;

FIG. 30 is a schematic view of the hydraulic version of the compression leg support in the second compressing stage;

FIG. 31 is a schematic view of the hydraulic version of the compression leg support in the third decompressing stage;

FIG. 32 is a side view of a linear actuator version of the compression leg support in place on a user's lower leg, with the compression leg support in a non-compressed position;

FIG. 33 is a side view of the linear actuator version of the compression leg support in place on a user's lower leg, with the compression leg support in a compressed position;

FIG. 34 is a back view of the linear actuator version of the compression leg support in a non-compressed position;

FIG. 35 is a front view of the linear actuator version of the compression leg support in a non-compressed position;

FIG. 36 is a side view of a biaxial sleeve version of the compression leg support in place on a user's lower leg, with the compression leg support in a non-compressed position;

FIG. 37 is a side view of the biaxial sleeve version of the compression leg support in place on a user's lower leg, with the compression leg support in a compressed position;

FIG. 38 is a front view of the biaxial sleeve version of the compression leg support;

FIG. 39 is a side view of the compression leg support with a front foot flap;

FIG. 40 is a side view of an alternate air pressure version of the compression leg support in a slightly open position;

FIG. 41 is a back view of the alternate air pressure version of the compression leg support;

FIG. 42 is an opposed side view of the alternate air pressure version of the compression leg support;

FIG. 43 is a front view of the alternate air pressure version of the compression leg support; and

FIG. 44 is a back view of the alternate air pressure version of the compression lag support in a fully open position.

Other advantages and features will be apparent from the following description and from the claims.

### DETAILED DESCRIPTION OF THE INVENTION

The devices and methods discussed herein are merely illustrative of specific manners in which to make and use this invention and are not to be interpreted as limiting in scope.

While the devices and methods have been described with a certain degree of particularity, it is to be noted that many modifications may be made in the details of the construction and the arrangement of the devices and components without departing from the spirit and scope of this disclosure. It is understood that the devices and methods are not limited to the embodiments set forth herein for purposes of exemplification.

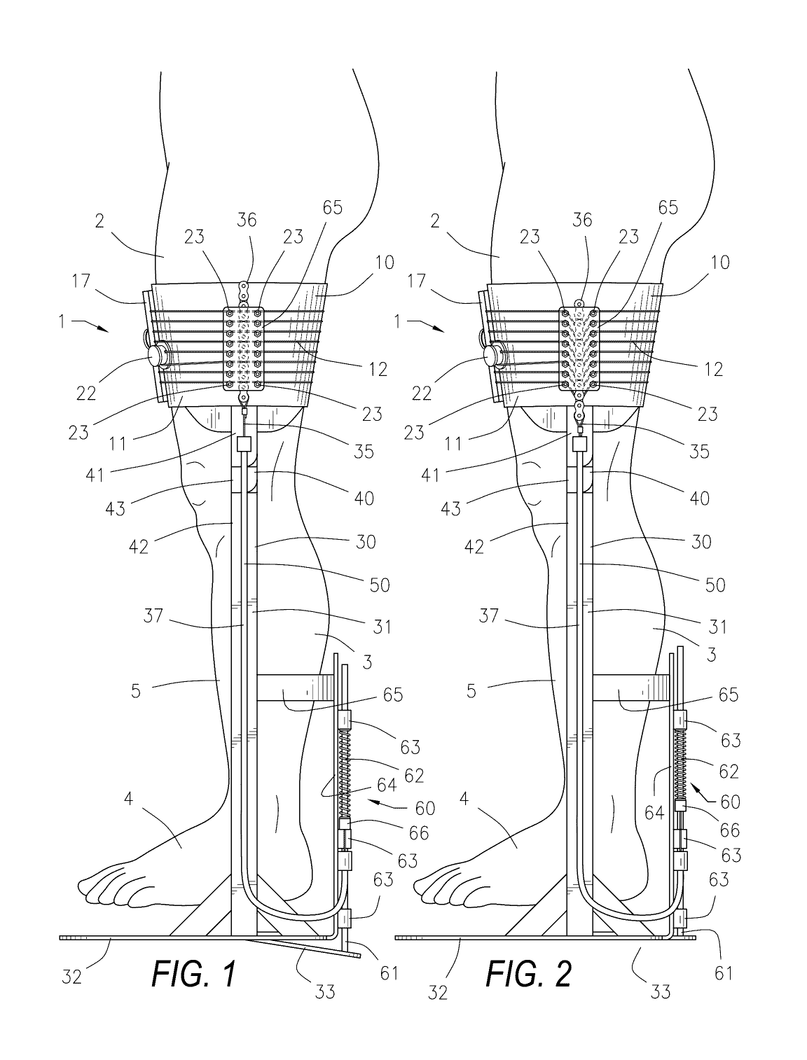

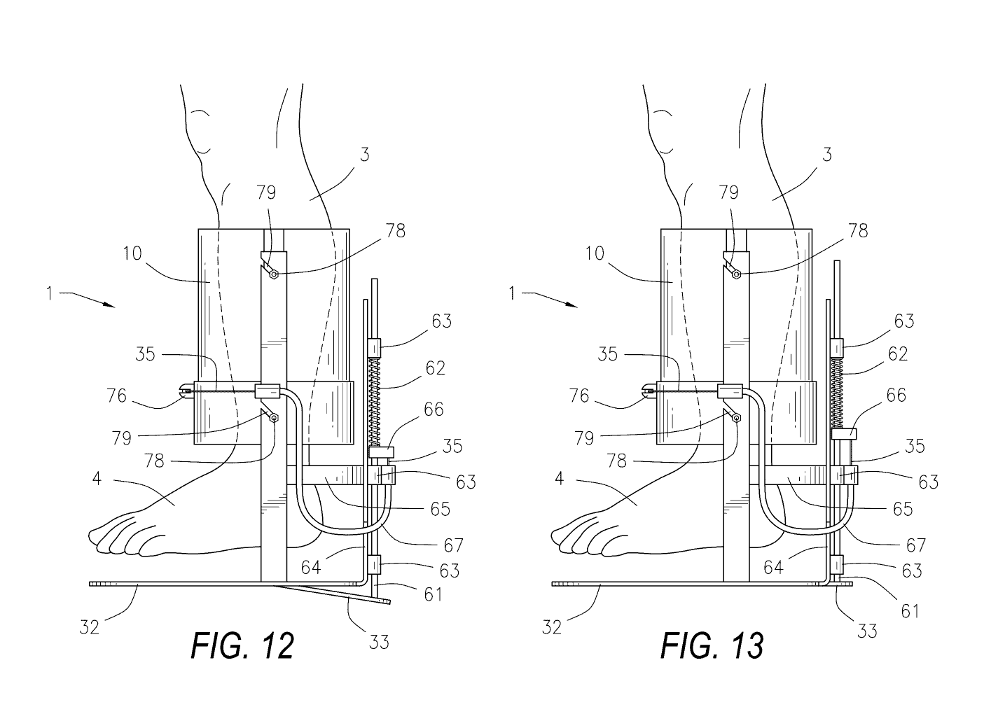

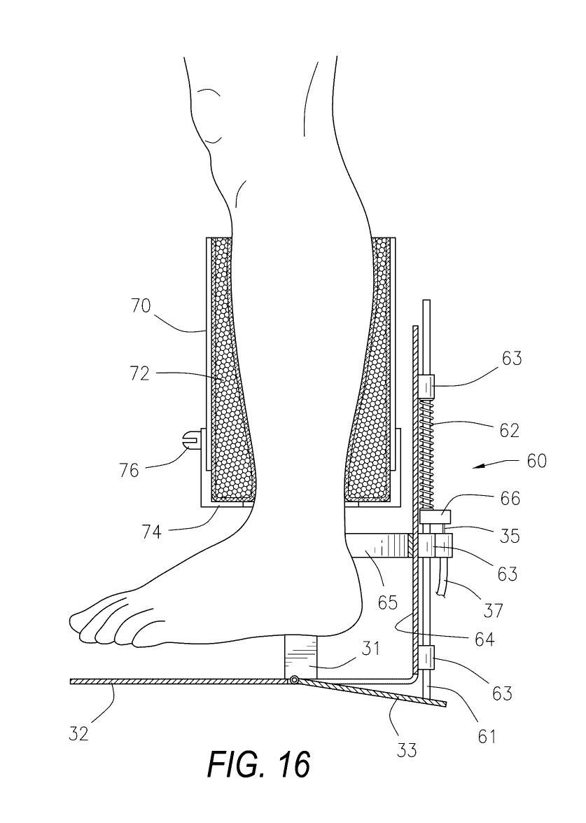

In general, in a first aspect, the invention relates to a leg support 1. The leg support 1 may wrap around a user's upper leg 2, as shown in FIGS. 1 through 3. The leg support 1 may surround the user's lower leg 3 and foot 4, and may optionally have a strap 5 that secures the leg support 1 to the user's lower leg 3. The strap may allow the user to control their center of gravity within the leg support 1 by moving their lower leg 3 forward or backward in the leg support 1, or the user may use the leg support 1 without the strap if desired. Alternately, the leg support 1 may wrap around the user's lower leg 3, as shown in FIGS. 12, 13, 16, 20, 21, 32, 33, 36, and 37. The leg support 1 may surround the user's foot 4. Whether the leg support 1 wraps around the user's upper leg 2 or lower leg 3, the leg support 1 may not touch the user's foot 4, either when the user has his or her weight on his or her leg or not.

The leg support 1 may comprise an upper assembly 10, which surrounds the user's upper leg 2 or lower leg 3 when in use, and a force conveyance assembly 30, which extends from the upper assembly 10 to beyond the user's foot 4 when in use. During use, the user may secure the upper assembly 10 around the user's upper leg 2 or lower leg 3 at a comfortable tension. The user may take a step, shifting his or her weight to the leg support 1. As the force conveyance assembly 30 extends beyond the user's foot 4, the force conveyance assembly 30 may come into contact with the floor before the user's foot 4. As a result of coming into contact with the floor, the force conveyance assembly 30 may cause the upper assembly 10 to tighten around the user's upper leg 2 or lower leg 3, as described below. Thus, a portion of the vertical shear pressure from the step may be transferred into horizontal compressional force around the user's upper leg 2 or lower leg 3, essentially causing the user's upper leg 2 or lower leg 3 to bear the user's weight. The increased compressive force may prevent the upper assembly 10 from slipping on the user's upper leg 2 or lower leg 3 and may keep the user's foot 4 from ever coming into contact with either the floor or the leg support 1. The result of this unique design may be zero pressure on the sole of the user's foot.

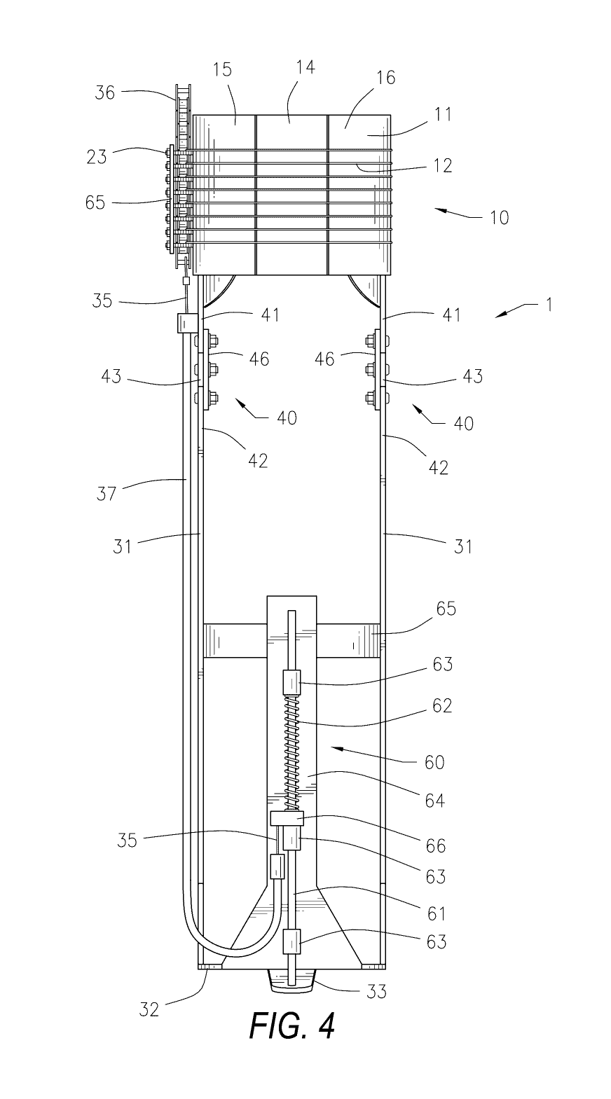

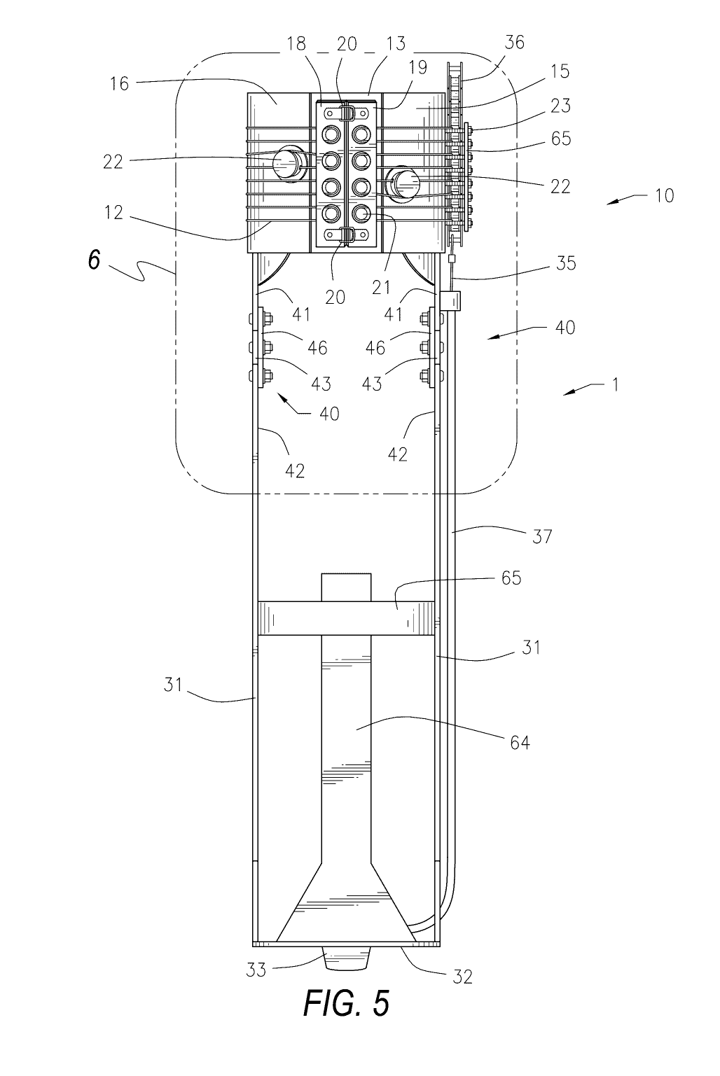

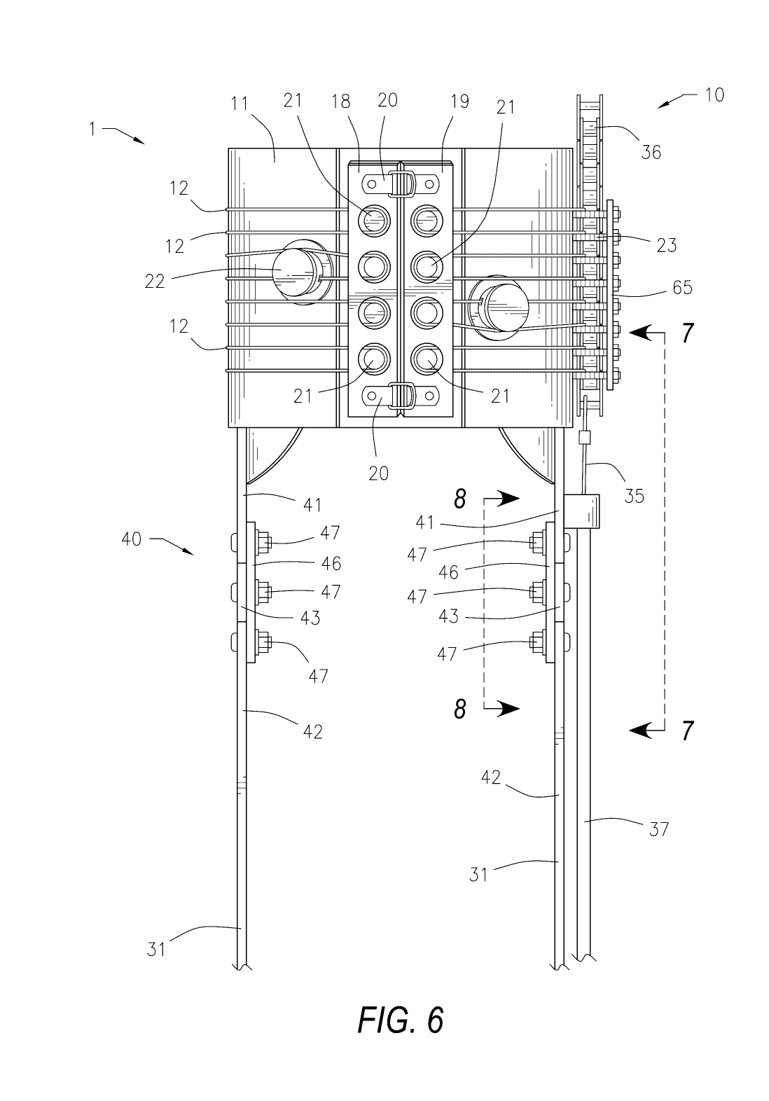

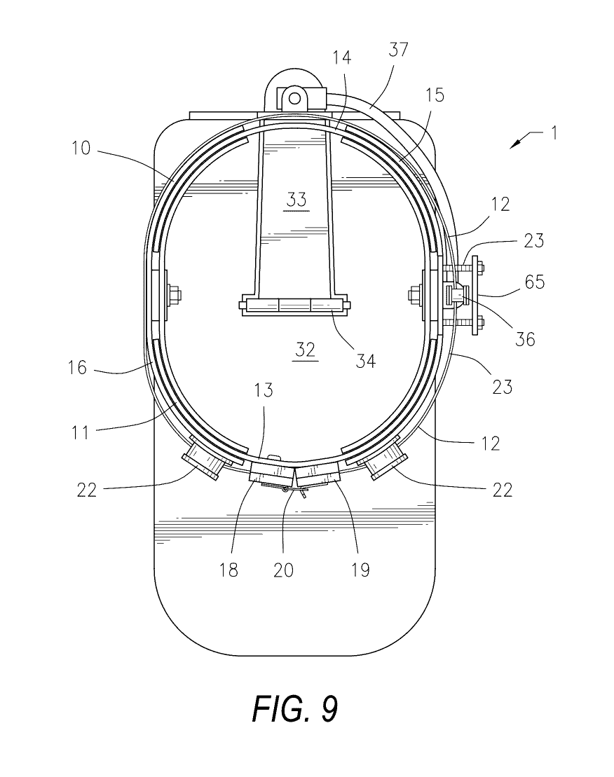

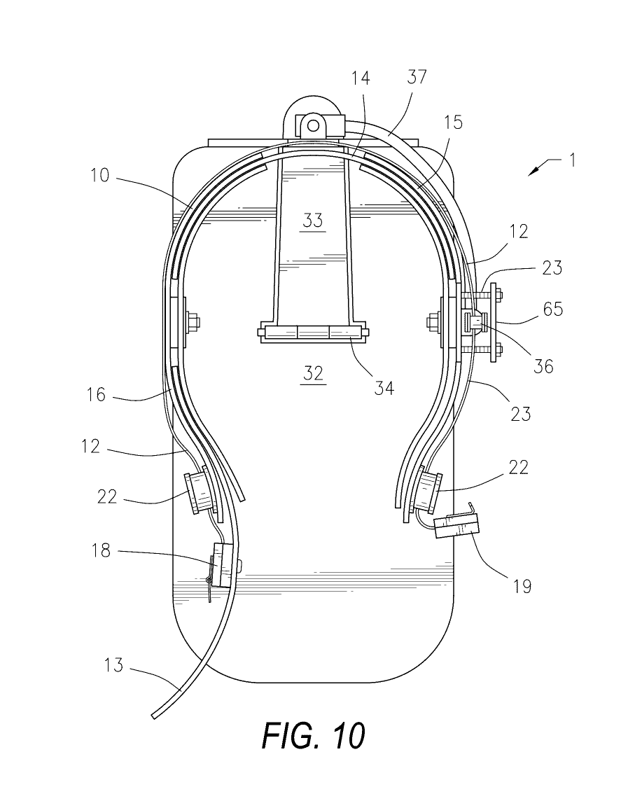

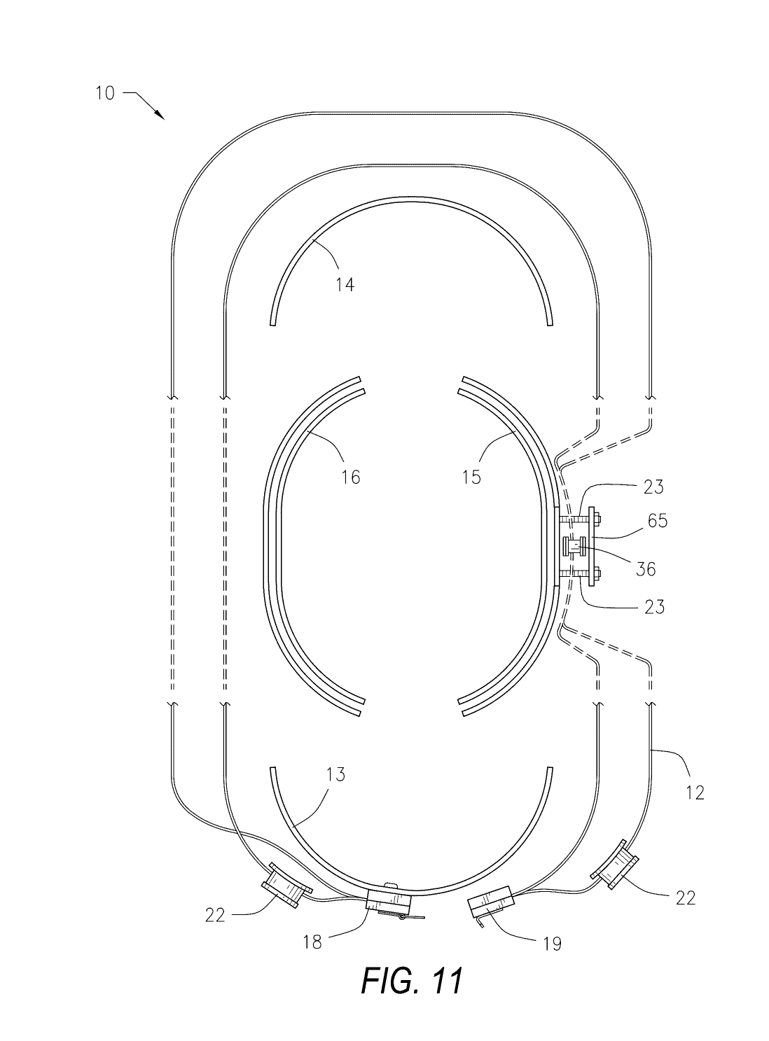

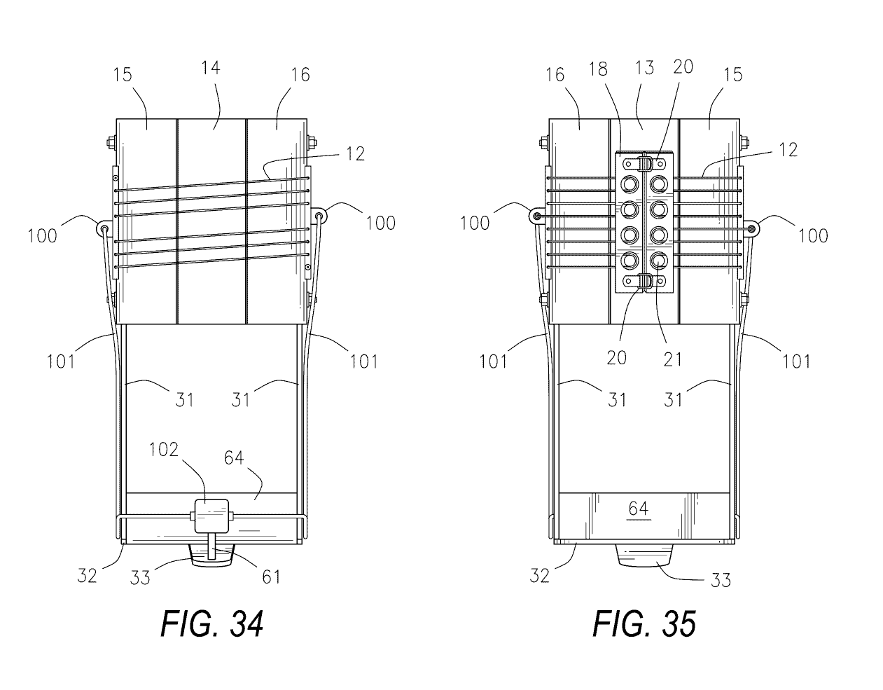

The upper assembly 10 may comprise a wrap 11 and at least one wire 12 substantially encircling the wrap 11, as shown in FIGS. 1 through 11 and FIGS. 32 through 35. The wrap 11 may be formed in such a way that it may tighten and loosen repeatedly during use without bunching. For example, as shown in FIGS. 9, 10, and 11, the wrap 11 may comprise a front section 13, a back section 14, a side section 15, and an opposing side section 16. The side sections 15 and 16 may each have a double layer, such that the sides of the front and back sections 13 and 14 may fit between the layers of the side sections 15 and 16 to form a cylinder. The cylinder may have a larger or smaller diameter depending on how much of the sections 13, 14, 15, and 16 overlap.

The wires 12 may fully encircle the wrap 11, which necessitates the user slipping the entire upper assembly 10 over his or her foot 4 and sliding it up into place on his or her upper leg 2 or lower leg 3 for use. Alternately, as shown in the drawings, the wires 12 may attach to a closure device 17, allowing the upper assembly 10 to be opened, placed around the user's upper leg 2, and closed for use. The closure device 17 may comprise a first closure side 18, which may attach to the front section 13 of the wrap 11, and a second closure side 19, which may be secured to the first closure side 18, such as via one or more attachment devices 20. The attachment devices 20 may be any desired attachment devices. The upper assembly 10 may open completely to go around the user's leg and may close completely to allow pressure adjustment. The design of the upper assembly 10 may allow pressure to be totally uniformly distributed around the user's leg with no one pressure point causing pain and discomfort.

The wire 12 may attach to both the first closure side 18 and the second closure side 19 such that the wire 12 extending between the first closure side 18 and the second closure side 19 may surround the wrap 11. The wire 12 may attach to the closure 17 in such a way that its tension may be adjusted. For example, the first closure side 18 and the second closure side 19 may each have a series of posts 21. A single wire 12 may be secured at one end to the first closure side 18 and may then extend to the second closure side 19, where it wraps around one of the posts 21. It may then extend back to the first closure side 18, where it wraps around another of the posts 21. It may extend backward and forward between the first and second closure sides 18 and 19 repeatedly before finally securing at its second end to the second closure side 19. A tensioning device 22 may be attached to the wire 12 such that tightening the tensioning device 22 may cause the wire 12 to slide along the posts 21, decreasing the circumference of the circle formed by the wire 12 and closure device 17. Loosening the tensioning device 22 may produce the opposite effect, assuming the tensioning device 22 was previously tightened such that there is slack wire 12 available. Alternately, multiple wires 12 and multiple tensioning devices 22 may be used. The use of multiple layers of wire 12 may allow a small vertical movement to translate to a large horizontal compression. For example, ½ inch vertical movement of the rod may equal approximately 2 inches of compression of the horizontal components.

The wires 12 may be held in place vertically by a plurality of anchors 23 with or without a plate 65, which may be attached to the side sections 15 or 16 of the wrap 11, depending on whether the leg support 1 will be used on the right or left leg. Preferably, anchors 23 may be attached on the side section 15 or 16 that is on the outer side of the user's leg, as opposed to the side adjacent the user's other leg. This is for the comfort of the user. The anchors 23 may be attached to the side section 15 or 16 such that they do not move relative to the side section 15 or 16, while the wire 12 may travel through or above the anchors 23 such that they may freely move laterally but may not move vertically.

The force conveyance assembly 30 may comprise two bars 31 extending downward from the upper assembly 20. One bar 31 may extend downward from each of the side sections 15 and 16 of the upper assembly 20. The bars 31 may be joined at their bottoms by a foot plate 32 extending therebetween. The foot plate 32 may be generally rectangular, foot-shaped, or any other desired shape and may be large enough to cover the bottom of the user's foot 4, as shown, or any other desired size. The bars 31 may be sufficiently long that the foot plate 32 is located below, but not touching, the user's foot 4 when the leg support 1 is in use. A flat piece 33 may be connected to the foot plate 32 in such a way that the distance between at least one side of the flat piece 33 and the foot plate 32 is variable. For example, as shown in the drawings, the flat piece 33 may be connected to the foot plate 32 via a hinge 34, with the back of the flat piece 33 at a variable distance from the foot plate 32. The flat piece 33 may be attached to the bottom of the foot plate 32 or may be located in an offset or opening in the foot plate 32 such that the flat piece 33 may be flush with the foot plate 32.

A cable 35 may be attached to the back of the flat piece 33, or whichever side of the flat piece 33 is a variable distance from the foot plate 32 if not the back, via a spring assembly 60, and may extend upward along one of the bars 31. The spring assembly 60 may comprise a rod 61 attached to or abutting the back of the flat piece 33, or whichever side of the flat piece 33 is a variable distance from the foot plate 32 if not the back, such that the rod 61 travels upward when the back of the flat piece 33 travels upward. A spring 62 may be mounted to the rod 61 such that the rod 61, and consequently the back of the flat piece 33, travels downward when pressure is removed from the flat piece 33. The cable 35 may be attached to the rod 61 such that moving the rod 61 upward pulls the cable 35. The cable 35 may connect to a chain assembly 36 between and aligned with the anchors 23. The wire 12 may travel through the chain assembly 36.

Specifically, the spring assembly 60 may comprise the rod 61 attached to or abutting the back of the flat piece 33 and extending upward. The rod 61 may be located to the rear of the leg support 1 and may be held in place vertically by a plurality of brackets 63 mounted to a support 64 extending vertically upward from the rear of the foot plate 32. The support 64 may be held in place via one or more cross pieces 65 attached to the bars 31. The rod 61 may move vertically freely through the brackets 63. The rod 61 may have a stop 66 mounted thereon, such that the stop 66 moves with the rod 61. The rod 61 may extend through the spring 62, which may be located above the stop 66 and below one of the brackets 63, such that when the rod 61 moves upward, the stop 66 forces the spring 62 to compress against the bracket 63. When the pressure is taken away from the flat piece 33, thus removing the upward pressure on the rod 61, the spring 62 may be allowed to decompress, pushing against the stop 66 and returning the rod 61 downward. The cable 35 may be attached to the stop 66 or elsewhere on the rod 61, then may travel initially downward through a cable sheath 37 before traveling upward along one of the bars 31. Thus, when the rod 61 moves upward, the cable 35 may be pulled downward along the bar 31.

When the user takes a step, the flat piece 33 may come into contact with the ground, forcing at least one side of the flat piece 33, such as the back as shown, upward. This may cause the rod 61 to move upward, pulling the cable 35. The cable 35 may be held in place laterally relative to the bar 31 by one or more eyelets or by the cable sheath 37. Thus, pulling the cable 35 may cause the wire 12 to be moved downward vertically at all of the points at which it extends through the chain assembly 36, which is likewise pulled downward by pulling the cable 35. This vertical motion of the wire 12 may be limited to the area between anchors 23, as they are secured to the wrap 11. This vertical motion may cause the wire 12 to tighten around the wrap 11. The more times the wire 12 extends substantially around the wrap 11, the greater the distribution of compressive force caused by the vertical motion of the cable 35. Thus, taking a step causes the upper assembly 10 to tighten around the leg of the user, allowing the user's upper leg 2 or lower leg 3, depending on where the upper assembly 10 is placed, to bear the user's weight rather than the user's foot 4.

When the user shifts his or her weight to the opposite foot, the pressure may be taken off the flat piece 33, allowing the spring 62 to urge the flat piece 33, rod 61, cable 35, and consequently the wire 12 to return to their normal positions. This increases the circumference of the upper assembly 10. This allows the upper assembly 10 to be tight enough to be effective when needed, but loose enough to be comfortable when not needed. The process repeats with each step.

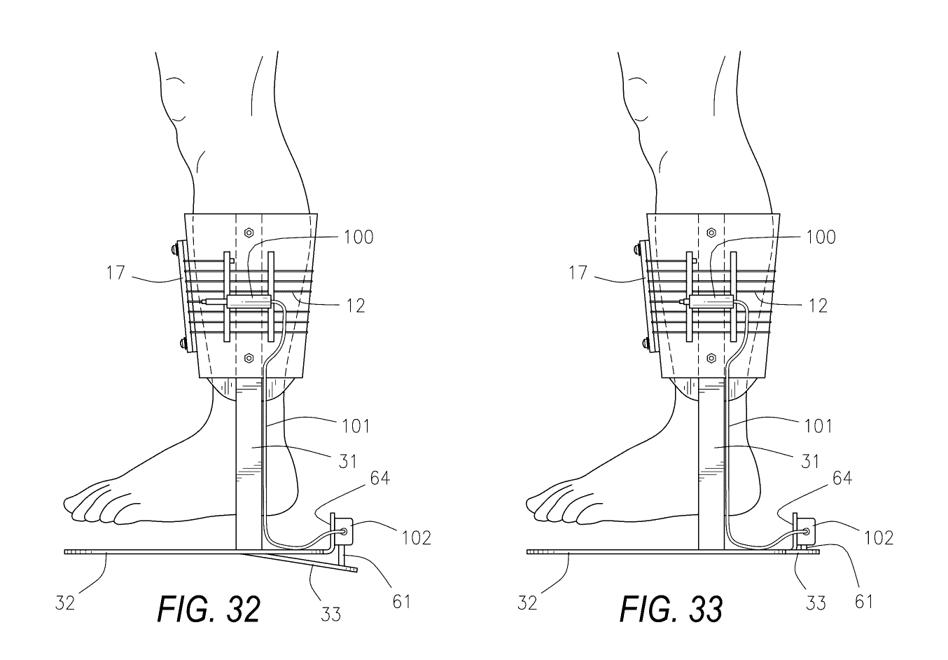

Alternately, as shown in FIGS. 32 through 35, the wire 12 may be tightened with one or more linear actuators 100. Each linear actuator 100 may be connected via a wire 101 to a switch 102 mounted to the foot plate 32 via the support 64. The upward movement of the rod 61 may close the switch 102, causing the linear actuator 100 to retract. The linear actuator 100 may be connected to the wire 12 such that retracting the linear actuator 100 may cause the wire 12 to tighten, as described above. Inversely, downward movement of the rod 61 may cause the linear actuator 100 to release, allowing the wire 12, and thus the entire upper assembly 10, to loosen. Linear actuators 100 may be located on each side of the upper assembly 10, thus simultaneously tightening and loosening two wires 12.

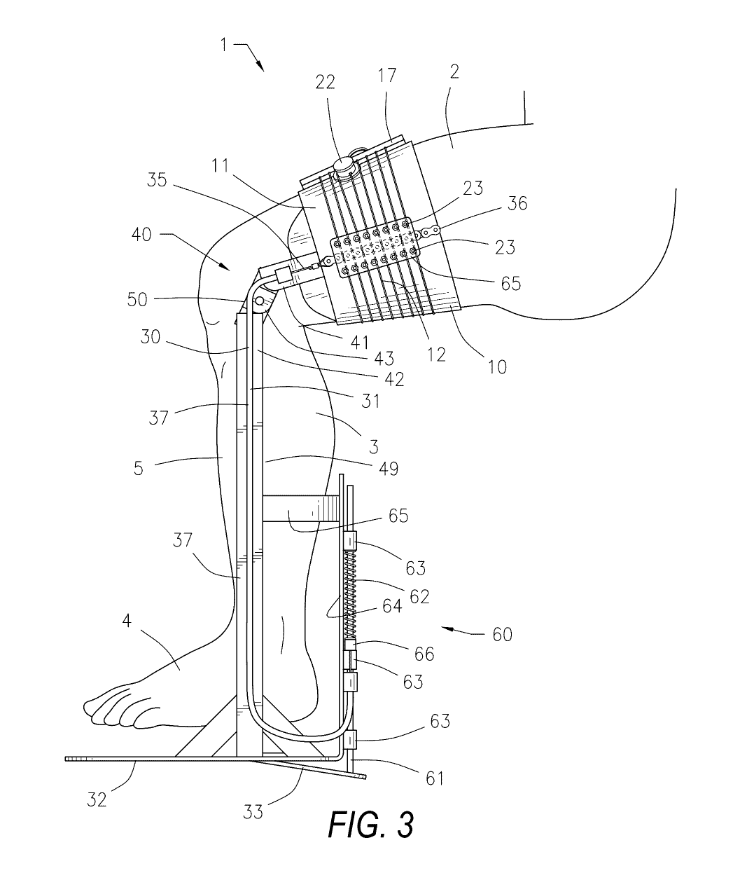

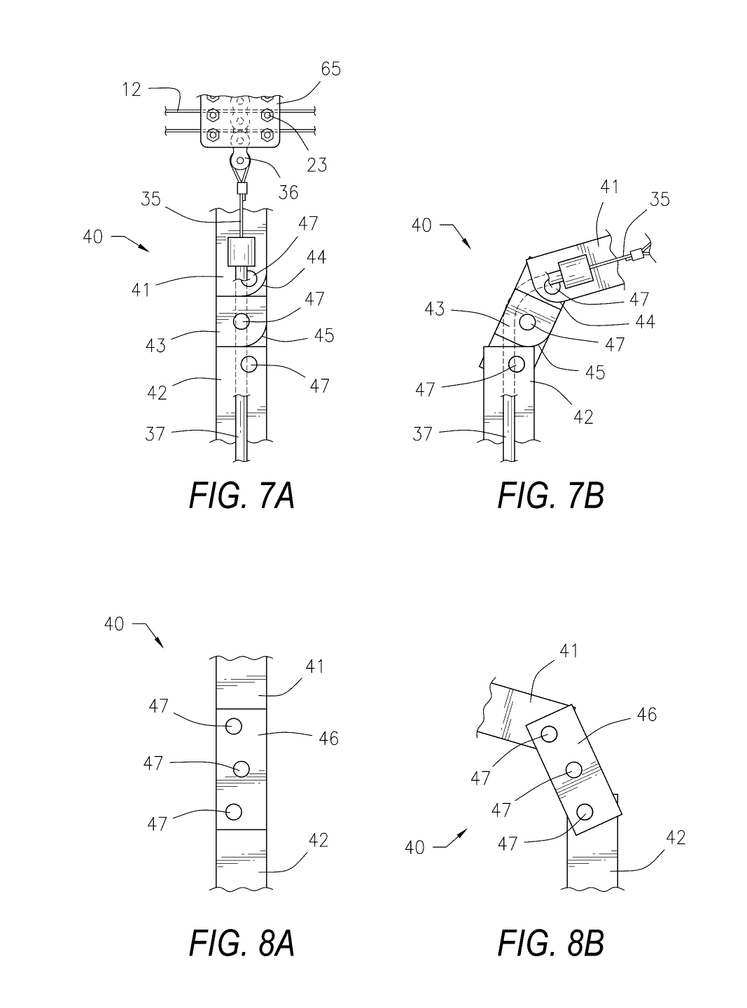

The upper assembly 10 may be worn around the user's upper leg 2, as shown in FIGS. 1, 2, and 3, or around the user's lower leg 3, as shown in FIGS. 12 and 13. If the upper assembly 10 is intended to be worn around the user's upper leg 2, the force conveyance assembly 30 may include further elements to allow the force conveyance assembly 30 to bend at the user's knee 6 for greater ease of use. For example, each of the bars 31 may have a hinge assembly 40. The outside of the hinge assembly is shown in FIGS. 7A and 7B, while the inside is shown in 8A and 8B. Each bar 31 may comprise an upper bar 41, a lower bar 42, and a middle piece 43 between the upper bar 41 and the lower bar 42. The upper bar 42 may have a rounded corner 44 adjacent the middle piece 43, and the middle piece 43 may have a rounded corner 45 adjacent the lower bar 42. The upper bar 41, middle piece 43, and lower bar 42 may be connected via a connector plate 46. The upper bar 41, middle piece 43, and lower bar 42 may be pivotally connected to the connector plate 46 via attachment devices 47, such as screws or bolts. The foregoing elements may allow the bars 31 to bend, as shown in FIGS. 3, 7B, and 8B.

The pivot points of the hinge assembly 40 may facilitate the arch bend of the user's knee. The center pivot point may be forward of the other two pivot points to allow the arch movement of middle piece 43 in relation to upper bar 41 and lower bar 42. The eccentric knee bend of the leg support 1 may allow constant pressure in all planes of movement.

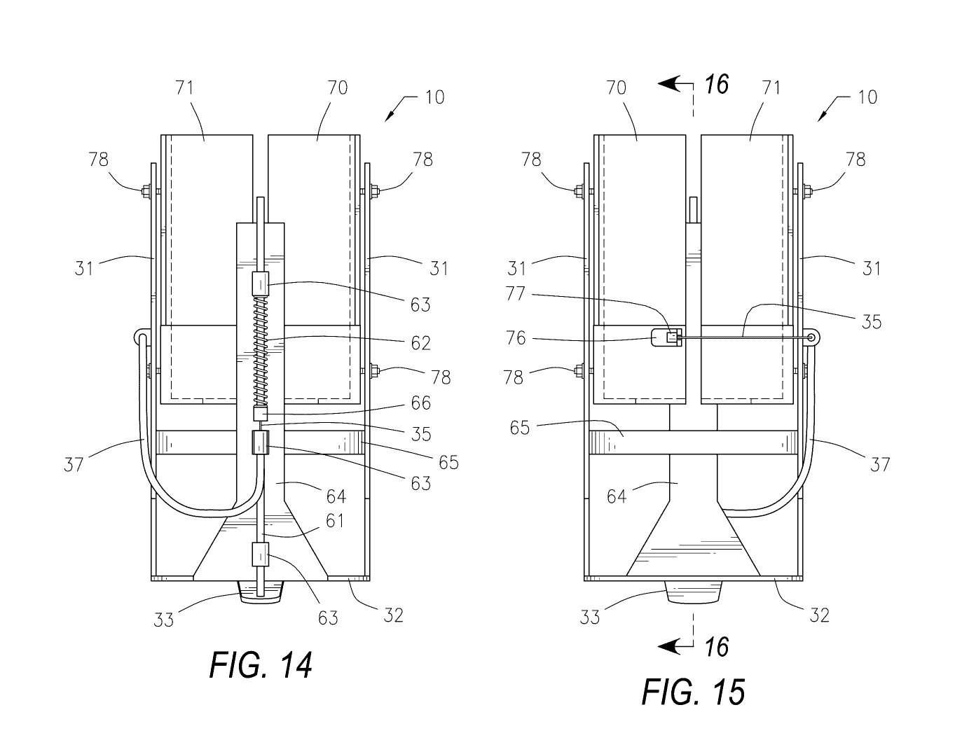

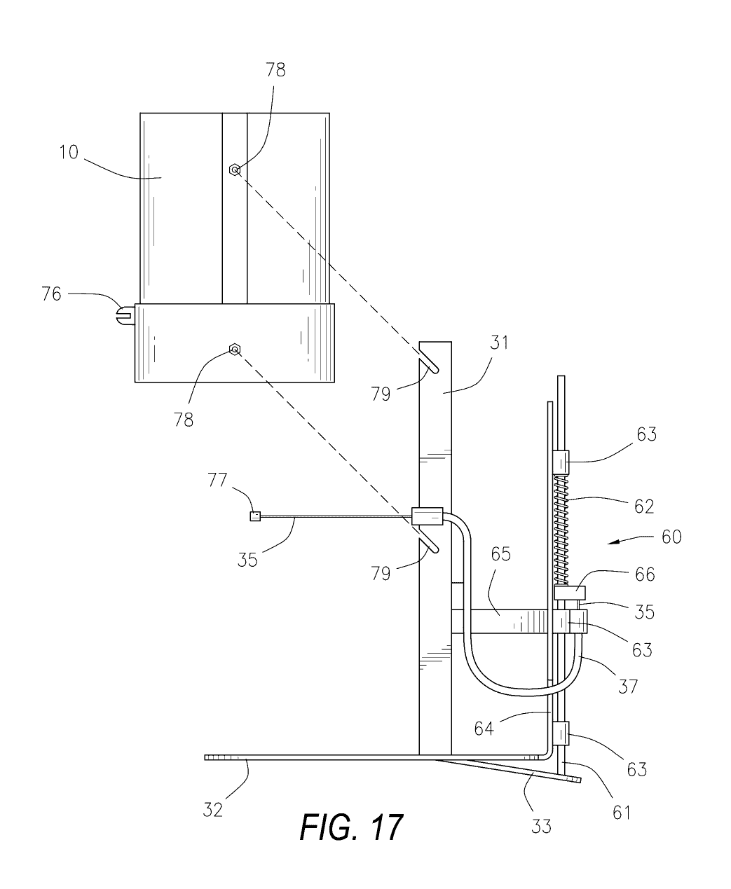

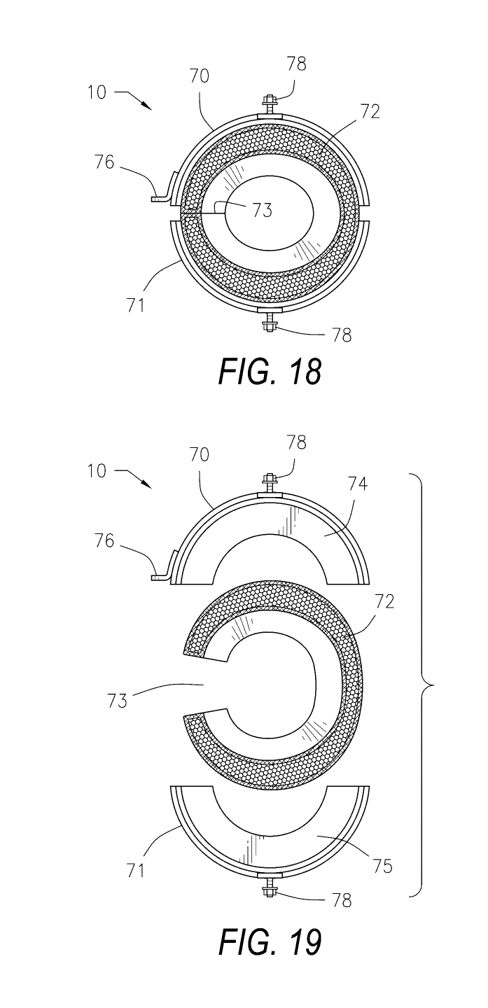

Alternately, as shown in FIGS. 12 through 19, the upper assembly 10 may have an adjustable circumference clamshell design, as shown in FIGS. 12 through 19. The upper assembly 10 may comprise a first half 70, a second half 71, and a form-fitting insert 72. The first half 70 and second half 71 together may be generally cylindrical, although the first half 70 and the second half 71 may not directly touch each other when the form-fitting insert 72 is in a relaxed state. The first half 70 and the second half 71 may be made of a generally rigid material. The first half 70 and the second half 71 may generally surround the form-fitting insert 72. The form-fitting material may be made at least partially of compressible material, such as foam. The form-fitting insert 72 may be generally cylindrical and may have a vertical break 73, allowing the user to deform the form-fitting insert 72 and place his or her leg through the break 73, thus placing his or her leg within the form-fitting insert 72. The first half 70 and the second half 71 may then be placed around the outside of the form-fitting insert 72. The first half 70 and second half 71 may each have a shelf 74 and 75, respectively, projecting inwardly laterally at their respective bottoms, allowing the form-fitting insert 72 to rest on the shelves 74 and 75. The outer diameter of the form-fitting insert 72 may be consistent, providing for a close fit with the first half 70 and the second half 71, but the inner diameter of the form-fitting insert 72 may be variable such that it may follow the contours of the user's leg. Indeed, the form-fitting insert 72 may be made specifically for the user by placing a mold around the user's leg and filling the mold with expanding foam to form the form-fitting insert 72, then cutting break 73 into the form-fitting insert 72 to remove it from the user's leg. The foam may be 4 psi expanding closed cell foam, or any other desired foam. The foam may have enough pressure when the form-fitting insert 72 is being made on the leg to compress fat and moisture, resulting in less movement necessary for the brace to secure horizontal pressure to the area desired.

As noted above, the first half 70 and the second half 71 together may be generally cylindrical, although the first half 70 and the second half 71 may not directly touch each other when the form-fitting insert 72 is in a relaxed state. Also as noted above, the form-fitting insert 72 may be made at least partially of compressible material, such as foam. The first half 70 may be brought toward the second half 71, thus reducing the diameter of the cylinder formed by the first half 70 and the second half 71 and at least partially compressing the form-fitting insert 72 within. When released, the form-fitting insert 72 may return to its relaxed state, separating or increasing the separation between the first half 70 and the second half 71.

The first half 70 and second half 71 may mount to the bars 31, where that the first half 70 mounts to one bar 31 and the second half 71 mounts to the other bar 31 such that the first half 70 and second half 71 align and are located between the two bars 31. The first half 70 and the second half 71 may each have one or more projections 78 and the bars 31 may have corresponding grooves 79 such that the projections 78 may fit within the grooves 79 to suspend the upper assembly between the bars 31. The grooves 79 may angle downward as shown such that gravity helps maintain the projections 78 within the grooves 79 during use.

The first half 70 may have a bracket 76 located along one of its sides adjacent the second half 71. The cable sheath 37 may run along the bar 31 upon which the second half 71 is mounted, and may terminated partway along the height of the second half 77 at approximately the same height as the location of the bracket 76 on the first half. Thus, the cable 35 may run from the spring assembly 60 up the bar 31, then laterally around a portion of the second half 71, as shown in FIGS. 12 and 13. The cable 35 may terminate in a stop 77 that fits within the bracket 76.

During use, the user may place his or her leg within the form-fitting insert 72 through the break 73, then may place the first half 70 and the second half 71 around the form-fitting insert 72. The user may attach the upper assembly 10 to the bars 31 by sliding the projections 78 into the grooves 79. The user may then place the stop 77 into the bracket 76. When the user takes a step, the flat piece 33 may come into contact with the ground, forcing at least one side of the flat piece 33, such as the back as shown, upward. This may cause the rod 61 to move upward, pulling the cable 35. This may pull the stop 77 against the bracket 76, pulling the first half 70 toward the second half 71. This may reduce the diameter of the cylinder formed by the first half 70 and the second half 71, thus compressing the form-fitting insert 72. This may increase the pressure of the form-fitting insert 72 on the user's leg. Thus, taking a step may cause the upper assembly 10 to tighten around the leg of the user, allowing the user's upper leg 2 or lower leg 3, depending on where the upper assembly 10 is placed, to bear the user's weight rather than the user's foot 4.

When the user shifts his or her weight to the opposite foot, the pressure may be taken off the flat piece 33, allowing the spring 62 to urge the flat piece 33, rod 61, and cable 35 to return to their normal positions. This takes the pressure off the upper assembly 10, allowing the form-fitting insert 72 to return to its relaxed state and the distance between the first half 70 and second half 71 to increase, thus increasing the circumference of the upper assembly 10. This allows the upper assembly 10 to be tight enough to be effective when needed, but loose enough to be comfortable when not needed. The process repeats with each step.

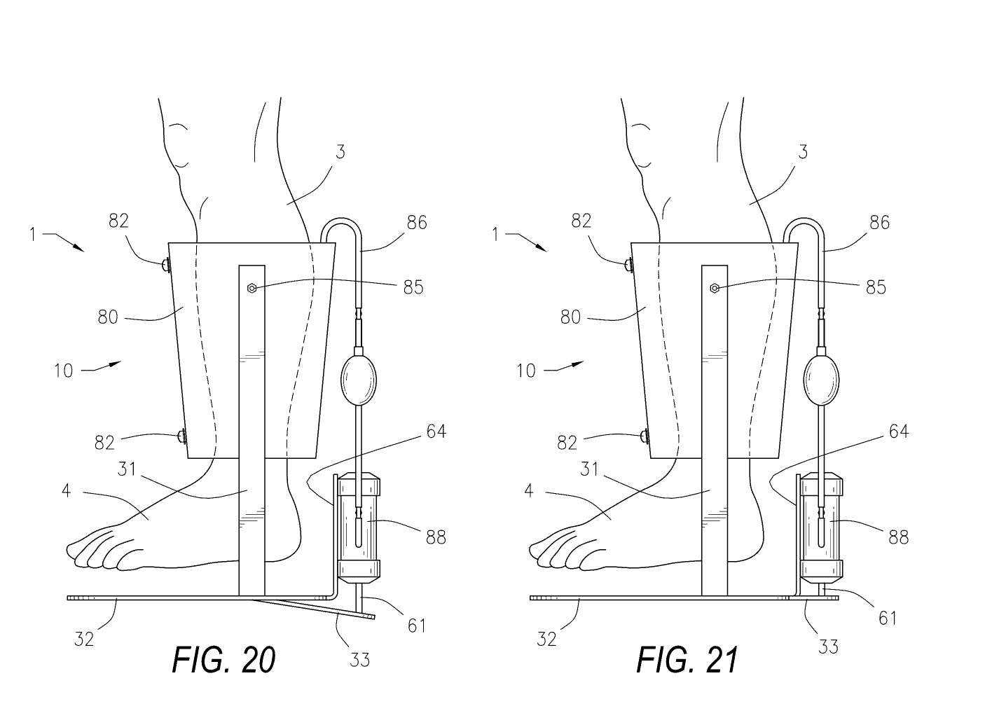

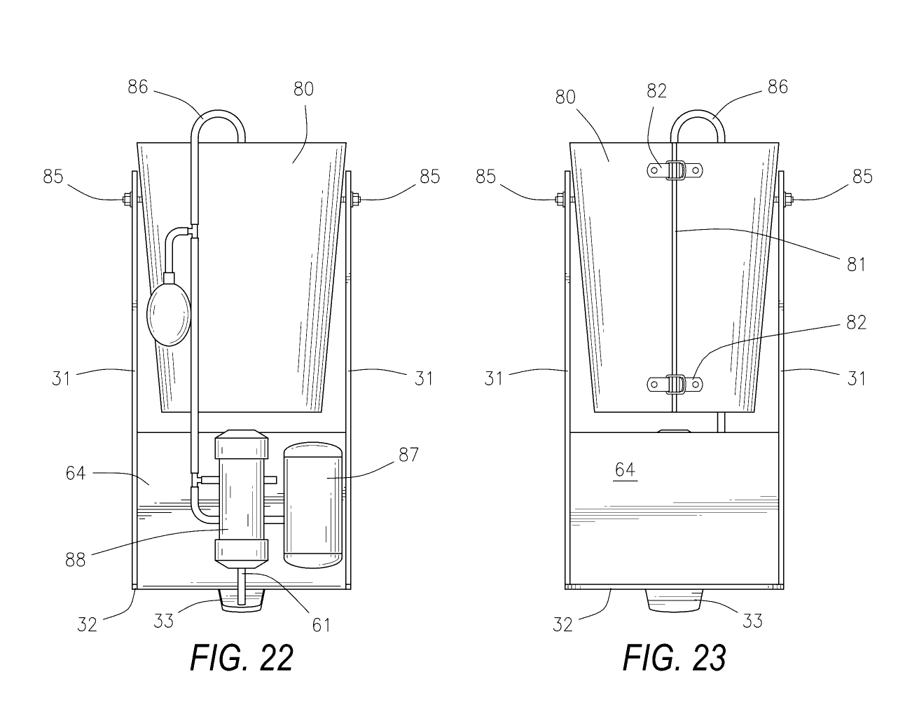

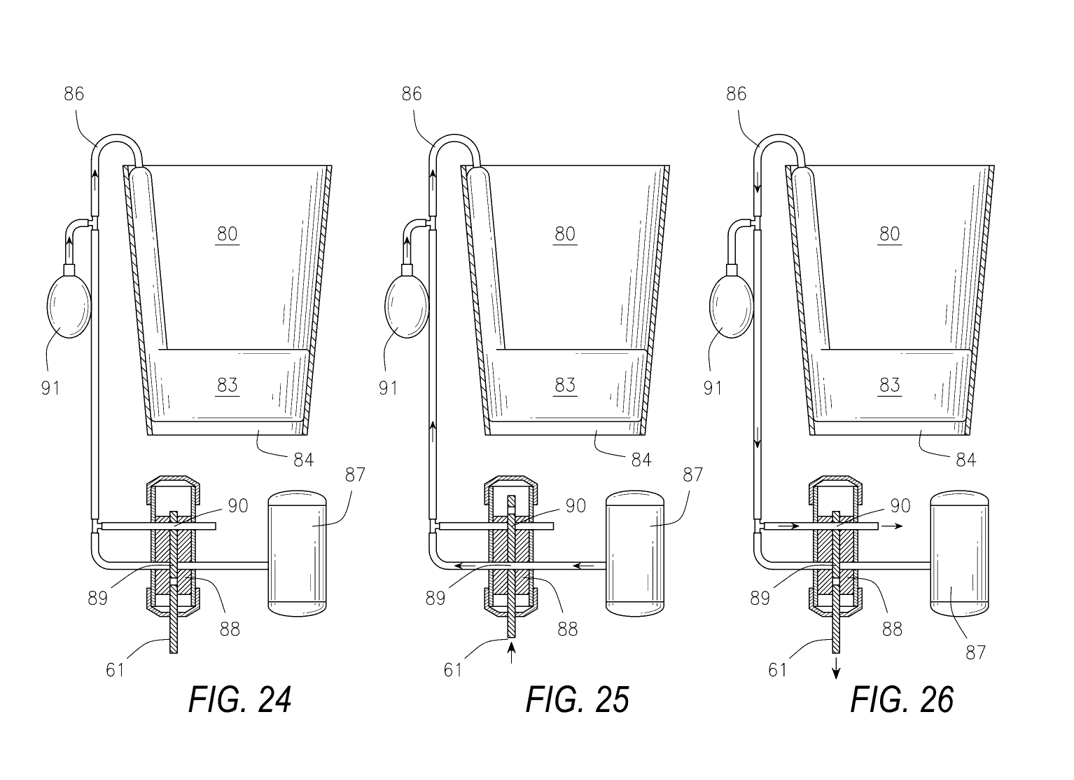

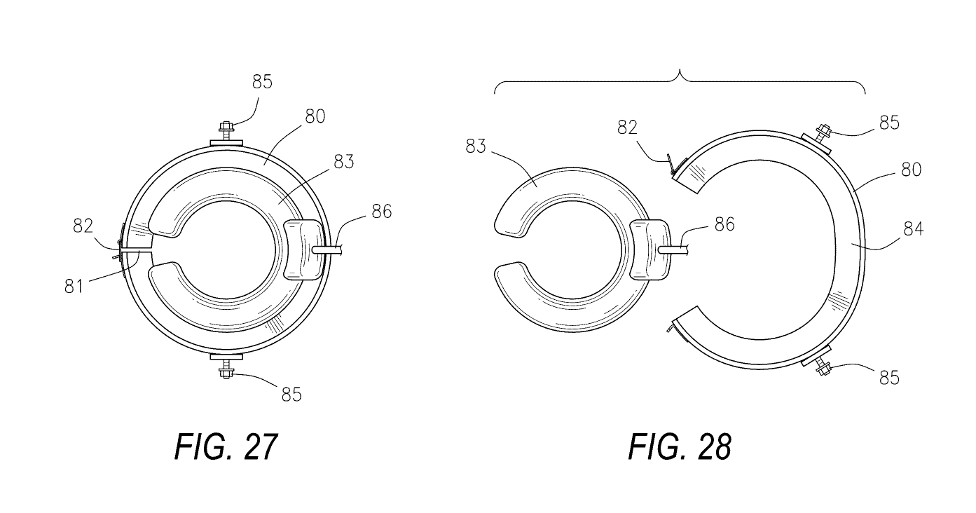

Alternately, as shown in FIGS. 20 through 31, the upper assembly 10 may comprise an outer shell 80, which may generally form a cylinder or conical frustum. The outer shell 80 may have a vertical break 81 and may be formed of a semi-rigid material such that the outer shell 80 may be deformed to place the user's leg within the upper assembly 10. The break 81 may be held closed by one or more latches 82. The outer shell 80 may house a bladder 83, which may have a c-shaped lateral cross section such that it at least partially, and preferably mostly or fully, surrounds the user's leg but allows a gap form placing the user's leg therein. The bladder 83 may be located low in the outer shell 80, such that it is located below the user's calf muscle when the upper assembly 10 is worn on the user's lower leg 3. Alternately, the bladder 83 may run the full height of the outer shell 80. The outer shell 80 may have a shelf 84 projecting inwardly laterally at its base, allowing the bladder 83 to rest on the shelf 84. The outer shell 80 may be mounted to the bars 31 via one or more attachment devices 85, such as screw and nut assemblies as shown or any other desired attachment devices, such that the upper assembly 10 is mounted between the bars 31.

The bladder 83 may be inflated either with compressed air or hydraulically. If the bladder 83 is inflated with compressed air, the bladder 83 may be connected via tubing 86 to a compressed air tank 87, as shown in FIGS. 20 through 28. The tubing 86 may connect to the tank 87 via a valve assembly 88. The tank 87 and valve assembly 88 may be mounted to the support 64. The valve assembly 88 may comprise the rod 61 such that when the rod 61 is moved upward by the flat piece 33, a valve 89 between the tubing 86 and the tank 87 is opened, allowing compressed air to travel from the tank 87 to the bladder 83, inflating the bladder 83. The valve assembly 88 may further comprise bleed valve 90 that is opened when the rod 61 is returned downward, allowing the bladder 83 to deflate. The bladder 83 may also be inflated manually by a bulb 91 attached to the tubing 86 via a check valve, allowing the user to partially inflate the bladder 83 to a comfortable level prior to use, thus decreasing the amount of air needed from the tank 87 to fully inflate the bladder 83.

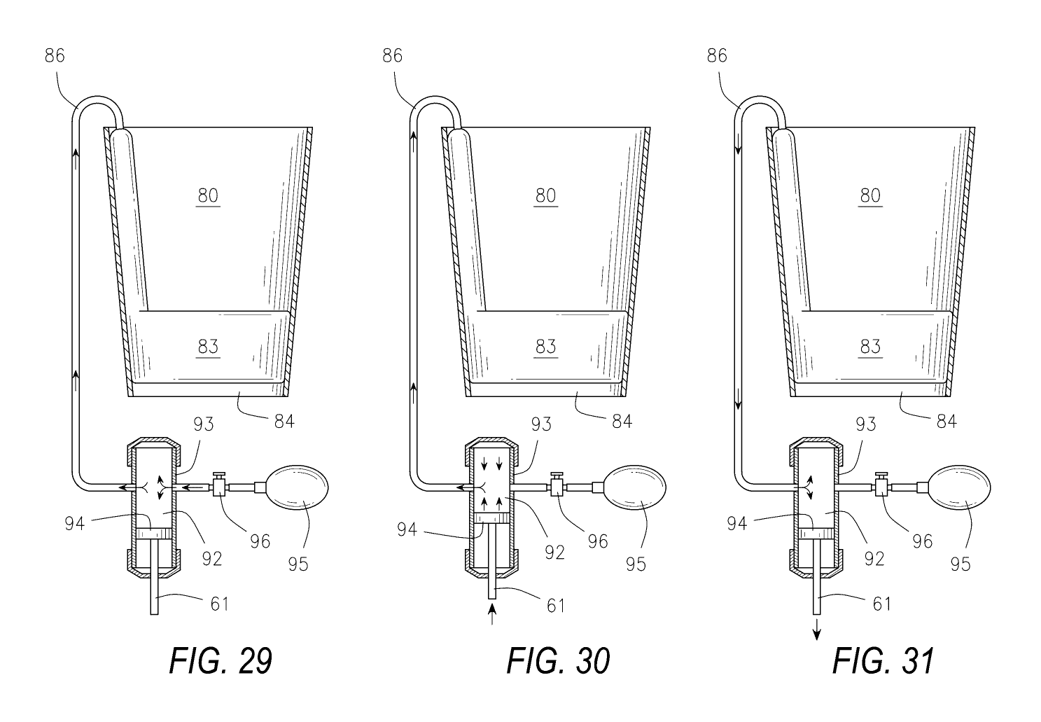

If the bladder 83 is inflated hydraulically, the bladder 83 may be connected via tubing 86 to a pump 92 mounted to the support 64, as shown in FIGS. 29 through 31. The pump 92 may be a reciprocating positive displacement pump, as shown. The pump 92 may comprise a cylinder 93 and a plunger 94 connected to the rod 61, such that upward motion of the flat piece 33 and the consequent upward motion of the rod 61 causes the plunger 94 to travel upward along the cylinder 93, reducing the volume thereof. This may cause fluid within the cylinder 93 to discharge into the tubing 86, where it travels to the bladder 83, thus inflating the bladder 83. Downward motion of the flat piece 33, and consequent downward motion of the rod 61, may cause the plunger 94 to travel downward along the cylinder 93, increasing the volume thereof and pulling fluid from the bladder 83, through the tubing 86, and back into the cylinder 93. A bulb 95 may be connected to the pump 92 via a valve 96, allowing the user to pressurize the system as desired.

Regardless of how the bladder 83 is inflated, inflating the bladder 83 may reduce the inner diameter of the upper assembly 10, thus tightening the upper assembly 10 against the user's leg. Thus, during use, the bladder 83 may be inflated when the user takes a step, tightening the upper assembly 10 and allowing the user's weight to be borne by the user's leg rather than the user's foot 4, and deflated when the user shifts to his or her other foot, loosening the upper assembly 10 to a comfortable level, as described above.

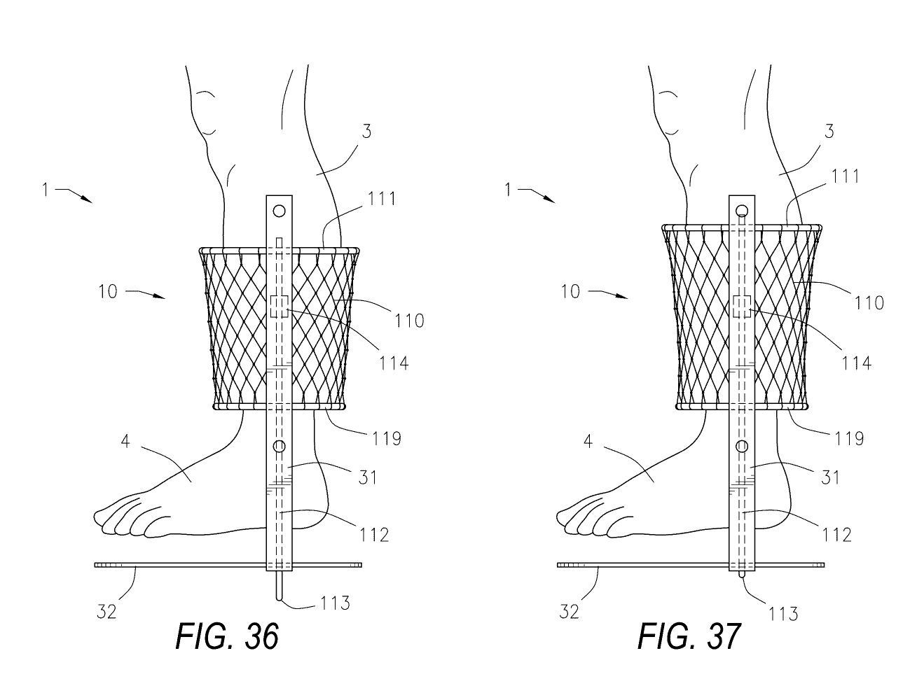

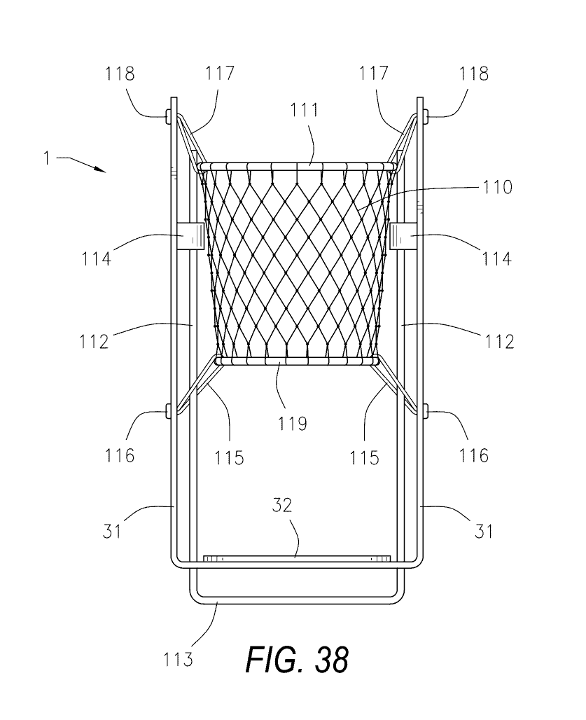

Alternately, the upper assembly 10 may comprise a biaxial sleeve 110, as shown in FIGS. 36 through 38. The sleeve 110 may reduce in circumference as it is stretched vertically. The sleeve 110 may surround the user's upper leg 2 or lower leg 3. The sleeve 110 may be suspended from its top by a ring 111, which may also surround the user's upper leg 2 or lower leg 3. The ring 111 may be connected via vertical supports 112 to a cross piece 113. The vertical supports 112 may run parallel to the bars 31, with the vertical supports 112 located adjacent to and just inside the bars 31 relative to the leg support 1 as a whole. The vertical supports 112 may be held in place laterally with respect to the bars 31 via a plurality of brackets 114, where the vertical supports 112 may move freely vertically within the brackets 114. The cross piece 113 may connect the bottoms of the vertical supports 112 and may be located below the foot plate 32. The bottom of the sleeve 110 may be connected to the bars 31, such as by ties 115 as shown, which may be held in place by attachment devices 116. Optionally, the top of the sleeve 110 may likewise be connected to the bars 31, such as by ties 117 and attachment devices 118. A ring 119 may hold the bottom of the sleeve 110 open and prevent the upward deformation of the bottom of the sleeve 110 when the top of the sleeve 110 is pulled.

During use, the user may place his or her leg within the sleeve 110. As the user takes a step, the cross piece 113 may come into contact with the floor or other walking surface prior to the foot plate 32 coming into contact with the same surface. This may cause the cross piece 113 to move upward relative to the foot plate 32, and consequently cause the vertical supports 112 to move upward relative to the bars 31. As the vertical supports 112 move upward, the ring 111 may move upward, pulling the top of the sleeve 110 upward relative to the bars 31. As the bottom of the sleeve 110 is held in place relative to the bars 31 by the ties 115, the sleeve 110 may stretch vertically. Because the sleeve 110 may be made of biaxial material, stretching the sleeve 110 vertically may cause its circumference to decrease. Thus, the sleeve 110 may tighten against the leg of the user, allowing the user's upper leg 2 or lower leg 3, depending on where the upper assembly 10 is placed, to bear the user's weight rather than the user's foot 4.

When the user shifts his or her weight to the opposite foot, the pressure may be taken off the cross piece 113, allowing the cross piece 113, vertical supports 112, ring 111, and consequently the top of the sleeve 110 to return to their normal positions. This allows the sleeve 110 to loosen. This allows the upper assembly 10 to be tight enough to be effective when needed, but loose enough to be comfortable when not needed. The process repeats with each step. The cross piece 113 may be located near the user's center of gravity, allowing for greater ease of movement and balance.

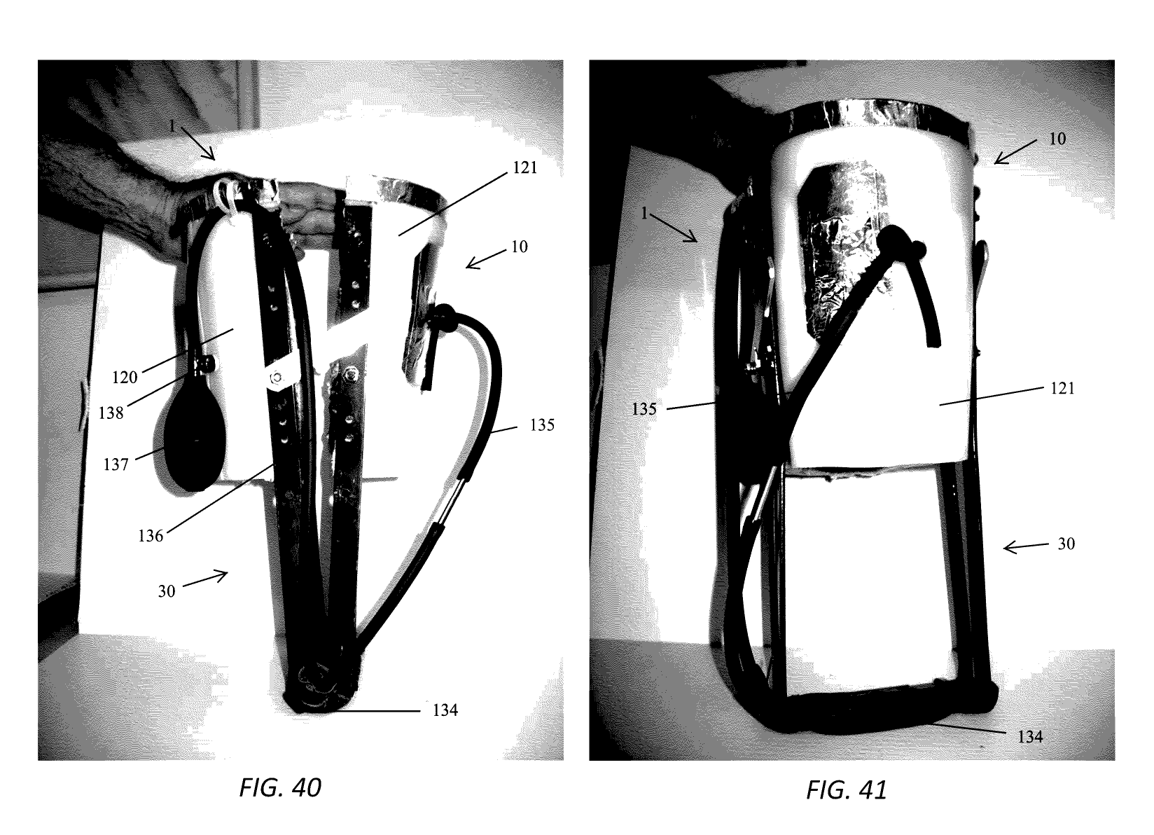

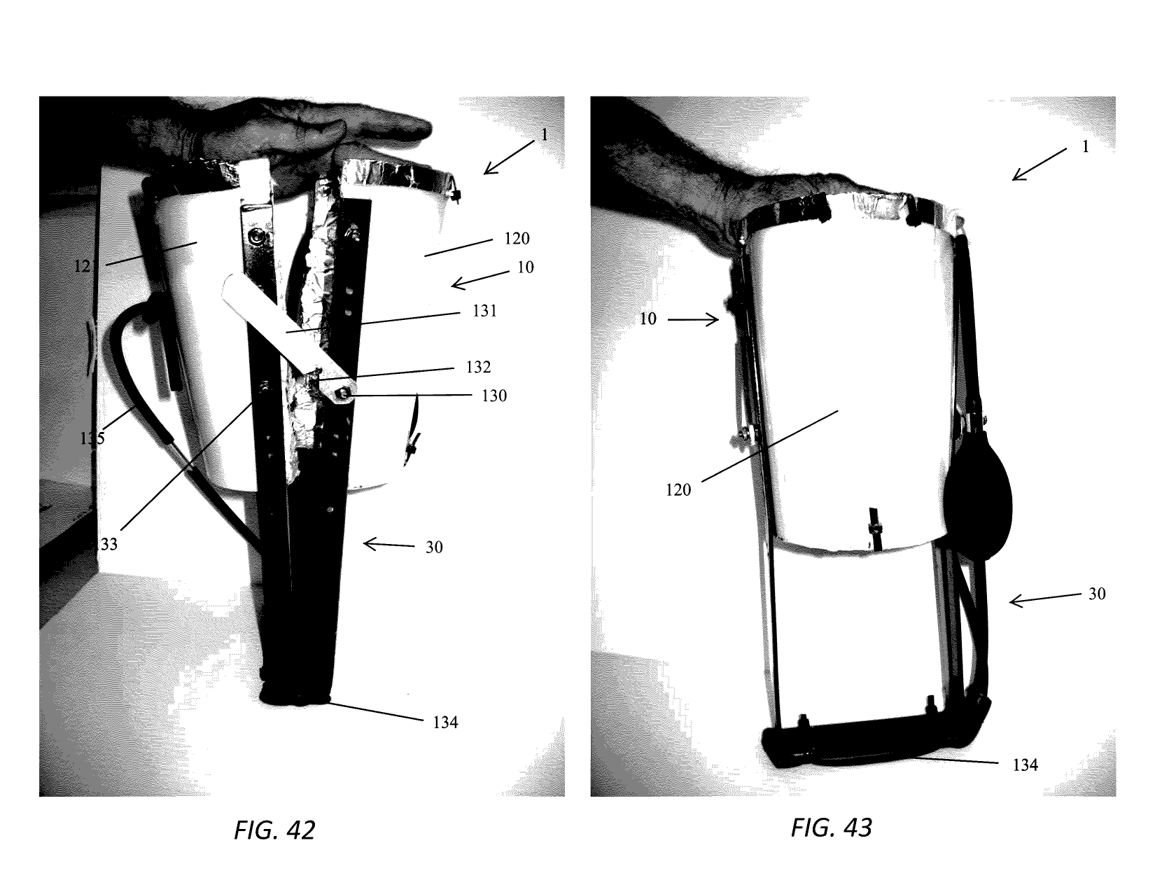

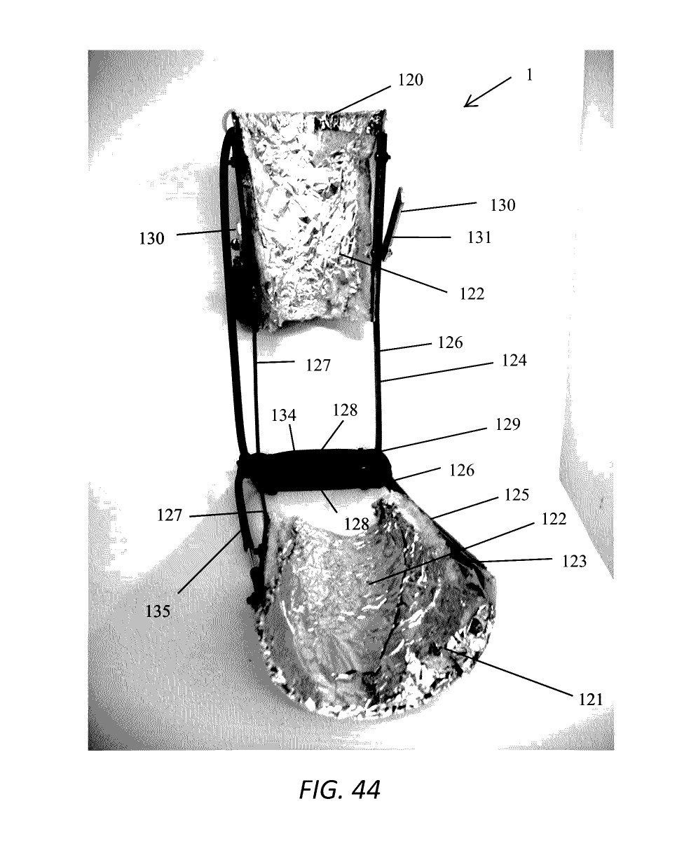

Alternately, as shown in FIGS. 40 through 44, the upper assembly 10 may comprise a front half 120 and a back half 121. The two halves 120 and 121 may join together to form a hollow cylinder or hollow conical frustum, sliced generally in half axially to form the two halves 120 and 121. The front half 120 and back half 121 may be made of generally rigid material. Inside each of the front half 120 and back half 121 may be a liner 122. Each of the liners 122 may cover at least a portion of the inside surface of the front half 120 or back half 121. Preferably, each of the liners 122 may cover substantially all of the inside surface of the front half 120 or back half 121. The liners 122 may be made of a form-fitting material such that the liners 122 contour to the user's leg 2 or 3. For example, the liners 122 may be made by placing the front half 120 and the back half 121 together over the user's leg 2 or 3 and inserting expanding foam therebetween, allowing the foam to expand and harden to form the liners 122. Sandwiched between the back half 121 and its liner 122 may be a bladder 123. Additionally or alternately, between the front half 120 and its liner 122 may be another bladder 123. Both of the bladders 123 may be expandable. When the upper assembly 10 is in a closed position and the bladders 123 are deflated, a gap may be maintained between the liner 122 of the front half 120 and the liner 122 of the back half 121, on either or both sides of the upper assembly 10.

The front half 120 and the back half 121 may connect via a pair of U-shaped brackets 124 and 125. The front bracket 124 may connect to the front half 120 while the back bracket 125 may connect to the back half 121. Each bracket 124 and 125 may have a right leg 126, a left leg 127, and a cross piece 128, where the right leg 126 and the left leg 127 are parallel and the cross piece 128 joins the bottoms of the right left 126 and the left leg 127 to form a generally U-shape. The two cross pieces 128 of the front bracket 124 and back bracket 125 may connect, such as via one or more hinges 129. The right leg 126 of the front bracket 124 may be connected to the front half 120 along the right side of the front half 120, while the left leg 127 of the front bracket 124 may be connected to the front half 120 along the left side of the front half 120. Likewise, the right leg 126 of the back bracket 125 may be connected to the back half 121 along the right side of the back half 121, while the left leg 127 of the back bracket 125 may be connected to the back half 121 along the left side of the back half 121. As such, the right leg 126 of the front bracket 124 and the right leg 126 of the back bracket 125 may be parallel and the left leg 127 of the front bracket 124 and the left leg 127 of the back bracket 125 may be parallel when the upper assembly 10 is in a closed position. The right legs 126 and the left legs 127 may be of sufficient length to ensure that the user's foot 4 does not touch the cross pieces 128 when the upper assembly 10 is in place on the user's leg 2 or 3.

To open the upper assembly 10, the front half 120 and the back half 121 may be separated, causing the cross pieces 128 of the front bracket 124 and the back bracket 125 to pivot relative to each other at the hinge 129. The upper assembly 10 may be held in a closed position by one or more closure devices 130. For example, a bar 131 may be pivotably attached to the right leg 126 of the front bracket 124. The bar 131 may have a slot 132 corresponding to a post 133 on the right leg 126 of the back bracket 125. Thus, the bar 131 may be pivoted such that the slot 132 engages the post 133 to lock the upper assembly 10 in the closed position, or pivoted such that the slot 132 disengages from the post 133 to unlock the upper assembly 10 from the closed position. A similar closure device 130 may additionally or alternately be located on the left legs 127 of the front and back brackets 124 and 125. Alternately, either or both closure device 130 may be any other desired closure device, such as hook and eye closure, bolt, etc.

The force conveyance assembly 30 may comprise a bladder 134 attached to and located below the cross pieces 128. The bladder 134 may be connected to the bladder(s) 123 such that bladder 134 is in fluid communication with bladder(s) 123. For example, bladder 134 may be fluidly connected to bladder 123 via tubing 135. As such, when a user wearing the leg support 1 takes a step, the bladder 134 may be pressed between the ground and the cross pieces 128 and air within the bladder 134 may be forced out of the bladder 134, along the tubing 135, and into the bladder 123. Inflating bladder 123 may cause the liner 122 to displace toward the user's leg 2 or 3, thus tightening the upper assembly 10 around the user's leg 2 or 3 and preventing the user's leg 2 or 3 from sliding down in the leg support 1 sufficiently for the user's foot 4 to touch the ground. When the user takes another step, shifting weight off of the leg support 1, the user's leg 2 or 3 may force the air in bladder 123 to travel along tubing 135 back to bladder 134, thus loosening the upper assembly 10 when high pressure is not needed and preparing for the next step.

The bladder 134 may also be connected to and in fluid communication via tubing 136 with a bulb 137 for adding air to the system and a valve 138 for releasing air from the system. Thus, the user can control the amount of air pressure in the force conveyance assembly 30 to a suitable, comfortable yet effective level.

The foregoing are examples of the leg support 1 and any of the alternatives discussed above may be combined as desired. The overriding points of the leg support 1 are that it includes an upper assembly 10 around the user's upper leg 2 and/or lower leg 3 and a force conveyance assembly 30 that causes the upper assembly 10 to tighten sufficiently when the user takes a step to keep substantially all pressure off the user's foot 4. Any of the upper assemblies 10 and force conveyance assemblies 30 described above or in U.S. Pat. No. 9,204,985 may be combined to achieve this result. For example, the vertical supports 112 and cross piece 113 shown in FIGS. 36 through 38 may connect to the upper assembly 10 shown in U.S. Pat. No. 9,204,985, but the upper assembly 10 may encircle the user's lower leg 3 rather than the user's upper leg 2, allowing the vertical supports 112 to connect directly with the upper assembly 10 without the hinge assembly 40. The upper assembly 10 may incorporate the form fitting insert 72 for a secure fit. Alternately, the compressed air tank 87 and valve assembly 88 may be employed, but mounted on the vertical supports 112 and actuated by the cross piece 113.

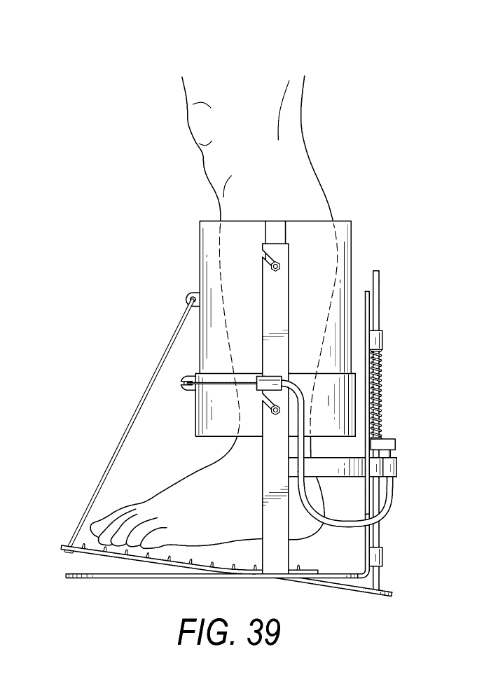

Regardless of which combination of alternatives comprise the leg support 1, the leg support 1 may further comprise a front foot flap 120, shown in FIG. 39. The front foot flap 120 may be flexible and may be attached on one end to the foot plate 32. The opposing end of the front foot flap 120 may be attached to the upper assembly 10 as shown, such as by cord 121 or other device. The purpose of the front foot flap 120 is to exert slight vertical pressure on the bottom of the foot in the toe area. This vertical pressure in the toe area sends a message to the user's brain that the forward rolling movement of the foot when a step is taken is now over. Therefore, the brain will shift weight to the other foot. This trick causes a quicker weight shift and less time of elevated pressure on the leg with the brace and foot flap than a conscious effort to do the same.

Whereas, the devices and methods have been described in relation to the drawings and claims, it should be understood that other and further modifications, apart from those shown or suggested herein, may be made within the spirit and scope of this invention.

What is claimed is:

1. A leg support comprising:
an upper assembly comprising:
a front half;
a back half; and
a bladder located within the front half or the back half;
a bracket comprising:
a right leg with a top connected to the upper assembly and a bottom;
a left leg with a top connected to the upper assembly and a bottom; and
a cross piece connecting the bottom of the right leg to the bottom of the left leg, where the cross piece has a bottom; and
a force conveyance assembly comprising a bladder attached to the bottom of the cross piece, where the bladder in the force conveyance assembly is in fluid communication with the bladder in the upper assembly such that pressing the bottom of the cross piece against a surface forces air within the bladder of the force conveyance assembly into the bladder in the upper assembly.

2. The leg support of claim 1 where the front half and the back half of the upper assembly are each elongate and have a semi-circular cross section such that the front half and the back half are capable of connecting to form a hollow cylinder or a hollow conical frustum.

3. The leg support of claim 1 where the upper assembly has a hollow cylindrical or hollow conical frustum shape.

4. The leg support of claim 1 where:
the front half and the back half of the upper assembly each have a right side and a left side;
the right leg of the bracket comprises a front right leg and a back right leg, where the front right leg is connected to the right side of the front half of the upper assembly and the back right leg is connected to the right side of the back half of the upper assembly;
the left leg of the bracket comprises a front left leg and a back left leg, where the front left leg is connected to the left side of the front half of the upper assembly and the back left leg is connected to the left side of the back half of the upper assembly; and
the cross piece comprises a front cross piece connecting the bottom of the front right leg to the bottom of the front left leg and a back cross piece connecting the bottom of the back right leg to the bottom of the back left leg, where the front cross piece is hingedly connected to the back cross piece.

5. The leg support of claim 1 where:
the front half of the upper assembly and the back half of the upper assembly each have an interior surface; and
the upper assembly further comprises:
a liner substantially covering the interior surface of the front half of the upper assembly; and
a liner substantially covering the interior surface of the back half of the upper assembly,
where the bladder is intermediate of either the interior surface of the front half and the liner substantially covering the interior surface of the front half or the interior surface of the back half and the liner substantially covering the interior surface of the back half.

6. The leg support of claim 5 where the liners comprise hardened expanding foam.

7. The leg support of claim 1 where the force conveyance assembly further comprises tubing fluidly connecting the bladder of the force conveyance assembly to the bladder of the upper assembly.

8. The leg support of claim 1 where the force conveyance assembly further comprises a bulb and a release valve fluidly connected to the bladder of the force conveyance assembly such that air pressure within the bladder is capable of being adjusted via the bulb and release valve.
